(19) United States
(12) Patent Application Publication
(10) Pub. No.: US 2013/0018325 A1
(43) Pub. Date: Jan. 17, 2013

(54) FLANGE EXTENDER FOR USE WITH AN INJECTION DEVICE AND METHOD OF ASSEMBLY

(71) Applicants: Eric Schiller, Westfield, NJ (US); Christina Joy Laskar, Cliffside Park, NJ (US); Mitali Aon, Princeton Junction, NJ (US); Richard Caizza, Vernon, NJ (US); Jon Bell, Waldwick, NJ (US)

(72) Inventors: Eric Schiller, Westfield, NJ (US); Christina Joy Laskar, Cliffside Park, NJ (US); Mitali Aon, Princeton Junction, NJ (US); Richard Caizza, Vernon, NJ (US); Jon Bell, Waldwick, NJ (US)

(73) Assignee: BRISTOL-MYERS SQUIBB COMPANY, PRINCETON, NJ (US)

(21) Appl. No.: 13/180,070

(22) Filed: Jul. 11, 2011

(51) Int. Cl. A61M 5/32; B23P 11/00; B29C 33/00; A61M 5/31

(52) U.S. Cl. 604/198; 604/187; 29/428; 264/299

(57) ABSTRACT

A flange extender for use with an injection device is described. The flange extender includes first and second flange extender pieces and a locking mechanism to lock the first and second flange extender pieces to one another. In one embodiment, the locking mechanism includes each piece having a locking finger extending from the piece and an aperture adapted to receive the locking finger. The first and second flange extender pieces extend in a direction transverse to a main axis of the injection device to thereby provide a surface area for manual manipulation of the injection device during an injection. The first and second flange extender pieces also attach to each other on opposite sides of the injection device.

### BACKGROUND

A. Field

This disclosure relates generally to injection devices and more particularly to a finger flange extender adapted to be attached to an injection device to enhance ease of administration of an injection, including self-administration of an injection.

B. Description of Related Art

A variety of devices for facilitating injection of medicament into a human or animal subject are known in the art. Some current injection devices include a safety device, such as a needle guard, coupled with a syringe to reduce the chances of accidental needle sticks and to facilitate safer disposal of the syringe. In general, the needle guard comprises a body and a shield which surround the syringe. One example of such a safety device and needle guard is shown and described in U.S. Pat. No. 6,623,459 and U.S. Pat. No. 7,255,689, both of which are incorporated by reference herein in their entireties.

A relatively large compressive force is exerted on the plunger of the syringe by the syringe barrel to provide for a tight, leak-proof seal. This quality of the seal, as well as the viscosity of the drug gauge of the needle, and self-injector's dexterity, can make the movement of the plunger difficult. Typically, a user applies three fingers on one hand during the injection process to overcome/exert these forces: the thumb which exerts a force on the plunger rod and the index and middle fingers which hold the barrel or needle guard of the syringe. Barrels and needle guards may include a finger grip or flange to facilitate controlling the rate of movement of the plunger relative to the syringe. Often, the finger grips are not easily handled by a user with low dexterity.

### SUMMARY

The following embodiments and aspects thereof are described and illustrated in conjunction with systems, tools and methods which are meant to be exemplary and illustrative, not limiting in scope.

In one aspect, a flange extender for attachment to an injection device is described. The flange extender includes first and second flange extender pieces and a locking mechanism to lock the first and second flange extender pieces to one another. The first and second flange extender pieces extend in a direction transverse to a main axis of the injection device to provide a surface area for manual manipulation of the injection device during an injection. The first and second flange extender pieces also attach to opposite sides of the injection device.

In another aspect, a flange extender for attachment to an injection device includes first and second flange extender pieces adapted to attach to opposite sides of the injection device, each piece having a locking finger extending from the piece and an aperture adapted to receive the locking finger and lock the first and second flange extender pieces to one another. The first and second flange extender pieces are of identical construction whereby each locking finger and aperture are spaced and configured such the locking finger of the first flange extender piece locks into the aperture of the second flange extender piece and the locking finger of the second flange extender piece locks to the aperture of the first flange extender piece to securely attach the first and second flange extender pieces to the injection device.

In yet another aspect, an injection assembly is disclosed. The injection assembly includes a pre-filled syringe having a barrel and a plunger, a safety shield surrounding the barrel of the syringe, the safety shield being movable between a retracted position and an extended position, a housing for the safety shield having a finger flange, and a flange extender comprising a first flange extender piece and a second flange extender piece located around the housing. The first flange extender piece is connected to the second flange extender piece to lock the flange extender around the housing.

In yet another aspect, a method of manufacturing a flange extender for an injection device is disclosed. The method includes using a mold cavity to form first and second flange extender pieces, the first and second flange extender pieces adapted to lock onto an injection device. The molded part includes a first feature forming a first locking mechanism and a second feature forming a second locking mechanism for mating with the first locking mechanism.

In yet another aspect, a method of assembling an injection device includes providing a syringe, directly attaching opposing pieces of a flange extender over the syringe, and engaging a locking mechanism to lock the flange extender around the syringe.

In addition to the exemplary aspects and embodiments described above, further aspects and embodiments will become apparent by reference to the drawings and by study of the following detailed description.

### BRIEF DESCRIPTION OF THE DRAWINGS

Exemplary embodiments are illustrated in the drawings. It is intended that the embodiments and figures disclosed herein are to be considered illustrative rather than restrictive.

FIG. 1 is a perspective view of an injection device including a flange extender of the present application;

FIG. 2 is a top view of the injection device shown in FIG. 1;

FIGS. 3a-3b are perspective views of a distal side of the flange extender shown in FIG. 1;

FIGS. 4a-4b are perspective views of a proximal side of the flange extender shown in FIG. 1;

FIG. 5 is a side view of one piece of the flange extender shown in FIG. 1;

FIG. 6 is a cross sectional view of a locking mechanism of the flange extender shown in FIG. 1;

FIG. 7 is a cross sectional view of one piece of the flange extender shown in FIG. 1;

FIG. 8 is a top view of the flange extender shown in FIG. 1;

FIGS. 9a-9b are perspective views of another embodiment of the flange extender of the present application; and

FIG. 10 is a perspective view of the flange extender shown in FIG. 1 in use.

### DETAILED DESCRIPTION

FIG. 1 shows an example injection device 100 having a flange extender 200 according to an embodiment of the present application. The injection device 100 includes a safety device such as a safety shield or needle guard 102 surrounding a syringe body (not shown). The injection device 100 has a main axis 101. In one example, the injection device 100 and needle guard 102 are in accordance with U.S. Pat. Nos. 6,623,459 and 7,255,689, which are both assigned to Safety Syringes, Inc., and which are incorporated by reference herein in their entireties. The needle guard 102 is movable between a retracted position in which the needle guard does not cover the needle and an extended position in which the needle guard covers the needle (not shown). The needle guard 102 may have a square or rectangular outer shape, as shown in FIG. 1. Alternatively, the needle guard 102 may have a different shape. The syringe includes a plunger 104. A user can apply a force to the plunger 104 to administer medication contained in the syringe.

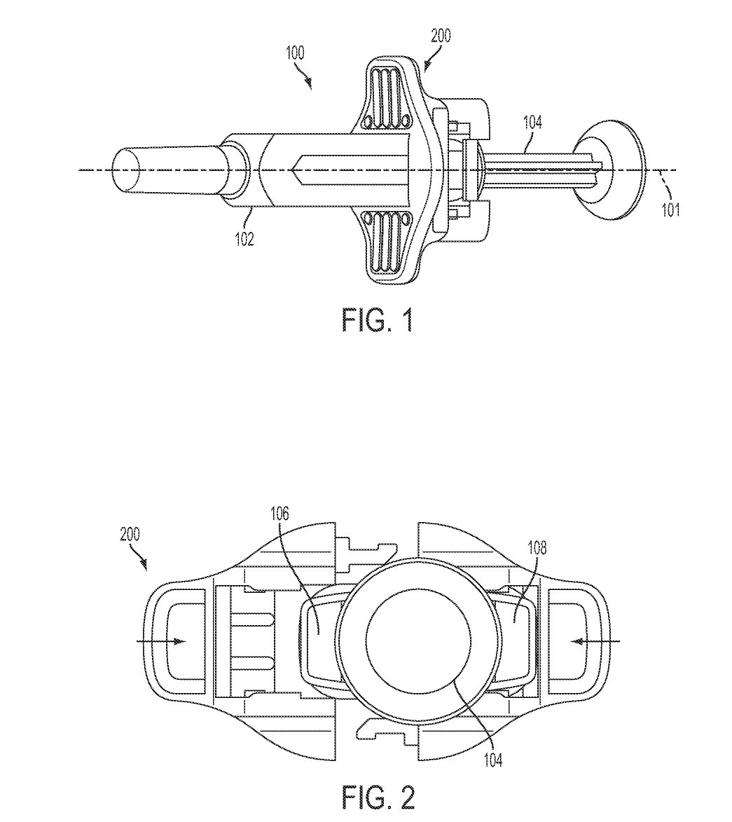

The needle guard 102 may also include flanges 106, 108 which can be seen in FIG. 2. The flanges 106, 108 facilitate controlling the rate of movement of the plunger 104 relative to the syringe. In one embodiment, as shown in FIG. 2, the flange extender 200 surrounds opposite sides of the flanges 106, 108 of the needle guard 102.

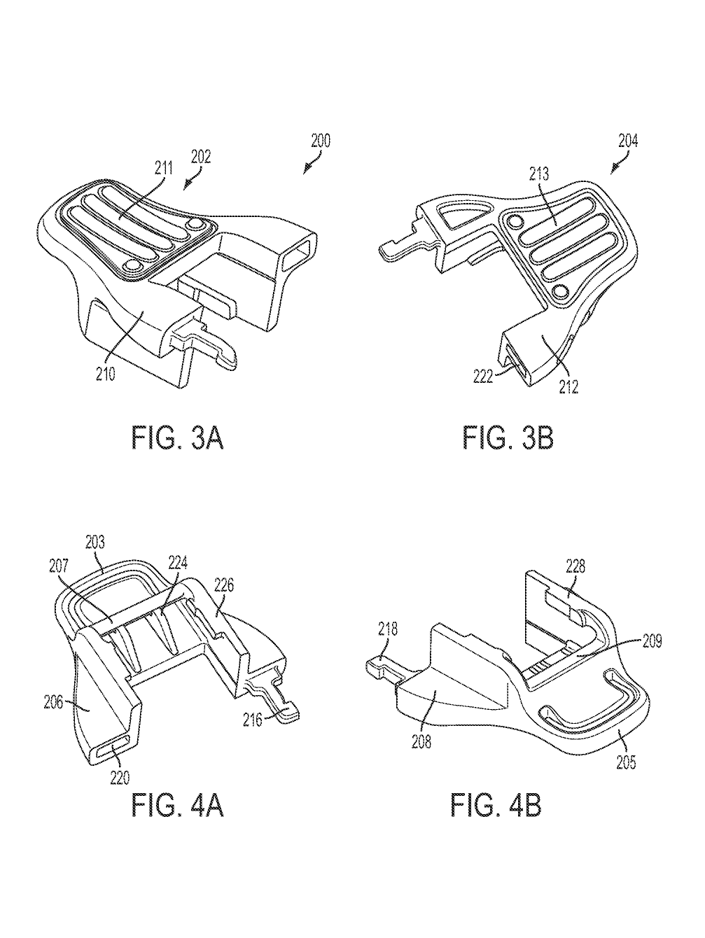

The flange extender 200 is secured around the needle guard 102, which is shown in detail in FIGS. 3-8. The flange extender 200 functions to enhance the ergonomics of injection by facilitating the gripping, handling, and comfort of a user of the injection device 100. In other examples, the flange extender 200 may be located around the body of any syringe instead of around a needle guard or safety shield. The flange extender 200 functions to substantially increase the surface area available to the user's index and middle fingers to grip and manipulate the injection device (as compared to the small flanges 106 and 108 of the underlying syringe assembly). The flange extenders thereby provide greater ability of a user to administer an injection, especially self-administration of an injection.

The flange extender 200 includes a first piece 202 and a second piece 204. The first and second flange extender pieces 202, 204 may extend in a direction transverse to the main axis 101 of the injection device 100 to provide a surface area for manual manipulation of the injection device during an injection and/or during aspiration of the syringe. The flange extender pieces 202, 204 are constructed in such a way that a user may grip the flange extender 200 left-handedly, right-handedly, or with both hands.

The flange extender 200 may be made of a plastic material, such as polycarbonate, for example. Alternatively, the flange extender 200 may be made of acrylonitrile butadiene styrene, polypropylene, high density polyethylene, and other rigid polymers or other materials with suitable rigidity.

The first and second flange extender pieces 202, 204 each include a proximal side 206, 208 and a distal side 210, 212, respectively. The proximal sides 206, 208 have a resting surface 207, 209 which accommodate the flanges 106, 108 of the needle guard 102, as shown in FIGS. 4a-4b. The distal sides 210, 212 include a soft material 211, 213, such as a thermoplastic elastomer (TPE), for example, to facilitate the gripping of the flange extender 200, as shown in FIGS. 3a-3b. The soft material 211 is of an injection moldable grade, and may have a Shore A hardness of between 30-50, for example. Other soft materials that may be used include silicone, natural or synthetic rubber, or foamed polyethylene, for example. Other possibilities exist as well. In the event that a material is selected for the soft material 211 that does not provide a chemical bond to the extender pieces 202 and 204, the soft material 211 can be attached to the flange extender pieces 202 and 204 by means of mechanical interlocks, cooperating locking mechanisms formed in the soft material and the extender pieces 202 and 204, or with the aid of adhesives. In preferred embodiments, the flange extender pieces are manufactured in a two shot molding method as described in further detail below.

The surface of the distal sides 210, 212 may be curved or textured to match the shape of a user's fingers and prevent slippage, which can be seen in FIG. 5. The proximal sides 206, 208 create a bearing surface for the flange extender pieces 202, 204 when aspirating the plunger to prevent excessive movement of the syringe in a proximal direction relative to the flange extender pieces. Further, the first and second flange extender pieces 202, 204 may each include a rounded peripheral end 203, 205. In other embodiments, the peripheral ends 203, 205 of the flange extender pieces 202, 204 may be shaped differently.

The flange extender pieces 202, 204 each include at least one inclined protrusion 224 on the proximal sides 206, 208. The inclined protrusion 224 facilitates assembly of the flange extender pieces 202, 204 by guiding the flanges 106, 108 of the needle guard 102 into the proper position. The inclined protrusions 224 also provide rigidity to the flange extender 200 when assembled.

The flange extender pieces 202, 204 each further include at least one outwardly extending wall 226, 228 on the proximal sides 206, 208. The outwardly extending walls 226, 228 align when the two flange extender pieces 202, 204 are locked together. The outwardly extending walls 226, 228 provide space for a locking mechanism 216, 218, 220, and 222 (described below). The outwardly extending walls 226, 228 also prevent the flange extender 200 from sliding along the needle guard 102 in the distal direction.

Of course, the design of the flange extender 200 is dictated by the design of the underlying injection device and may vary depending upon the particular design of the syringe and/or safety mechanism with which it is used.

As described above, the first and second flange extender pieces 202, 204 attach to each other on opposite sides of the flanges 106, 108 of the needle guard 102. Alternatively, the first and second flange extender pieces 202, 204 may attach to the needle guard 102 at a position rotated 90 degrees from the flanges 106, 108. In this embodiment, the internal construction of the flange extender 200 would be different than that which is described above to accommodate the underlying injection device. The placement of the flange extender 200 on the needle guard 102 does not obscure visualization of the fluid fill level within the syringe or leading edge of stopper prior to injection when viewed from one side of the device. In addition, the flange extender 200 does not impede visualization of the syringe label information.

The first flange extender piece 202 locks to the second flange extender piece 204 to secure the flange extender 200 onto the needle guard 102 by a locking mechanism 216, 220. In one embodiment, as shown in FIGS. 3-7, the locking mechanism 216, 220 includes at least one finger and corresponding aperture located on each flange extender piece 202, 204. In this embodiment, the first and second flange extender pieces 202, 204 have the same physical configuration. It should be understood that other locking mechanisms may also be used.

The first flange extender piece 202 has a first locking finger 216 and a first aperture 220, and the second flange extender piece 204 has a second locking finger 218 and a second aperture 222. The first locking finger 216 of the first extender piece 202 mates with the second aperture 222 of the second extender piece 204, and the second locking finger 218 of the second extender piece 204 mates with the first aperture 220 of the first extender piece 202. Although the locking fingers 216, 218 and apertures 220, 222 are shown as being centrally located on the flange extender pieces 202, 204, it should be understood that the locking mechanism may be located anywhere on the flange extender pieces, such as on the proximal 206, 208 or distal 210, 212 side surfaces, for example. Further, the locking fingers 216, 218 may face inwardly toward a recess 230, which is described below.

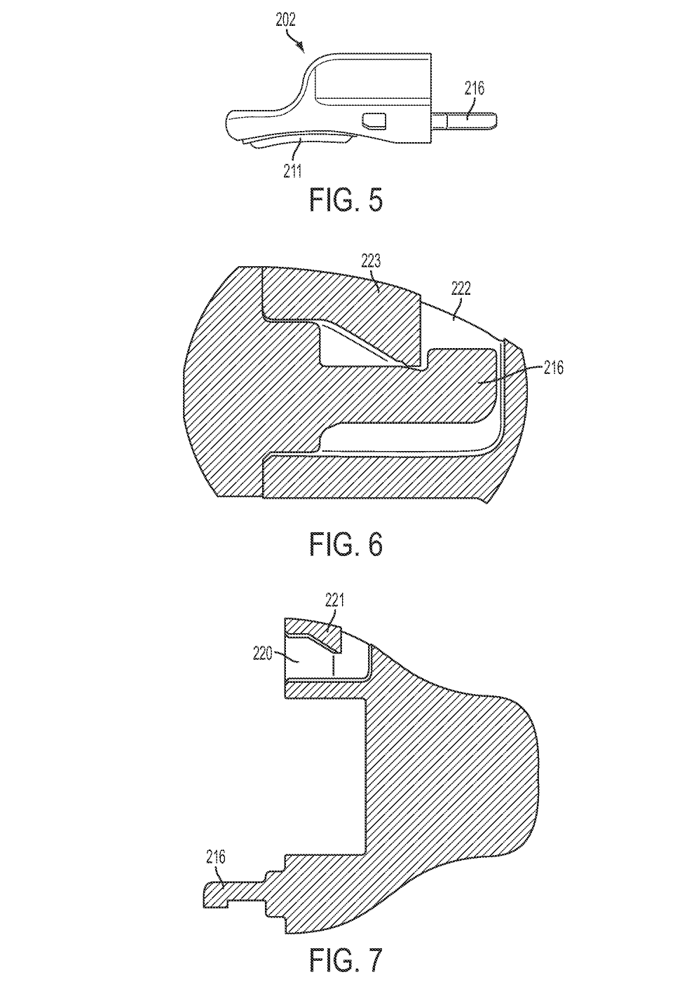

As shown in FIG. 6, the locking fingers 216, 218 are snap-fit into the apertures 220, 222. Each aperture 220, 222 includes a protuberance 221, 223 upon which the corresponding locking finger 216, 218 rests in the locked position. Alternatively, the locking fingers 216, 218 may be secured to the apertures 220, 222 in any suitable manner. The apertures 220, 222 may have a tapered lead-in radius or chamfer, as shown in FIG. 7, to facilitate alignment of the locking fingers 216, 218 with the apertures 220, 222.

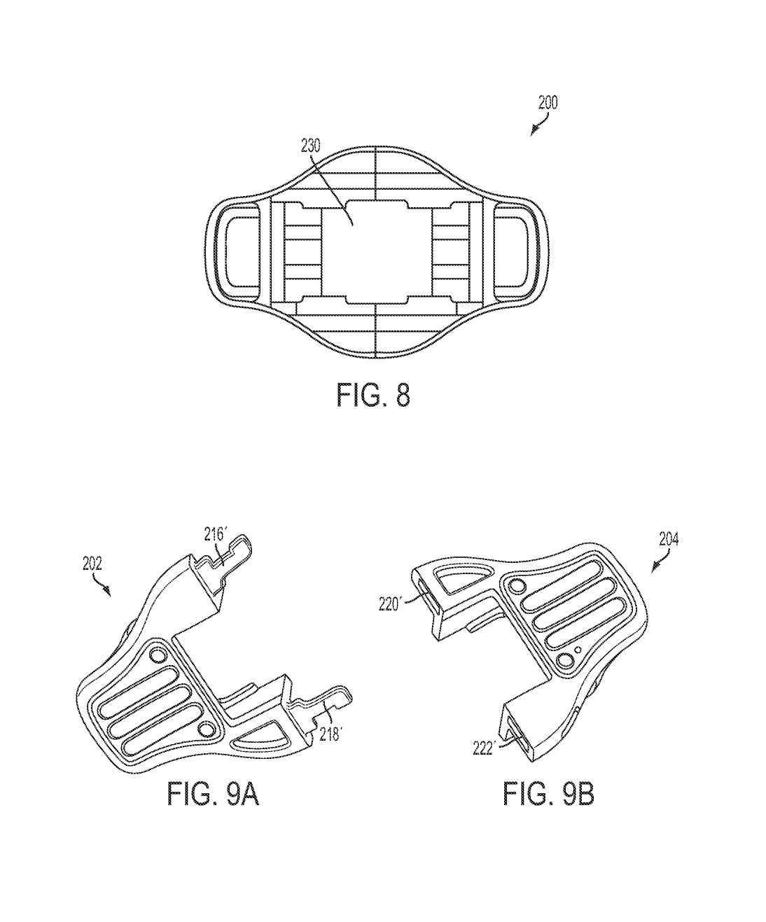

Referring to FIG. 8, when the first and second flange extender pieces 202, 204 are connected, a recess 230 is formed through which the needle guard 102 is received. In one embodiment, the recess 230 may have a rectangular shape to accommodate the rectangular outer shape of the needle guard 102. Alternatively, the needle guard 102 and recess 230 may be any other shape, as long as they are complimentary to create a secure connection. Additional shapes of the recess 230 may include circular, cut flange (straight sides with rounded edges), round, elliptical, or square, for example.

In another embodiment, shown in FIGS. 9a-9b, the first flange extender piece 202 has two locking fingers 216′ and 218′, and the second flange extender piece 204 has two apertures 220′ and 222′. The locking fingers 216′, 218′ mate with the apertures 220′ and 222′ to lock the flange extender 200 onto the needle guard 102. The locking fingers 216′, 218′ can be snap-fit into the apertures 220′, 222′ in a manner similar to that described above with respect to FIG. 6. Alternatively, the locking fingers 216′, 218′ may be secured to the apertures 220′, 222′ in any suitable manner. Although the locking fingers 216′, 218′ are shown to face outwardly, it should be understood that one or both of the locking fingers 216′, 218′ may face inwardly toward the recess 230.

Alternatively, any suitable locking mechanism can be used to secure the first extender piece 202 to the second extender piece 204, such as, for example, an adhesive, ultra-sonic welding, press fit, or a loop and hook arrangement.

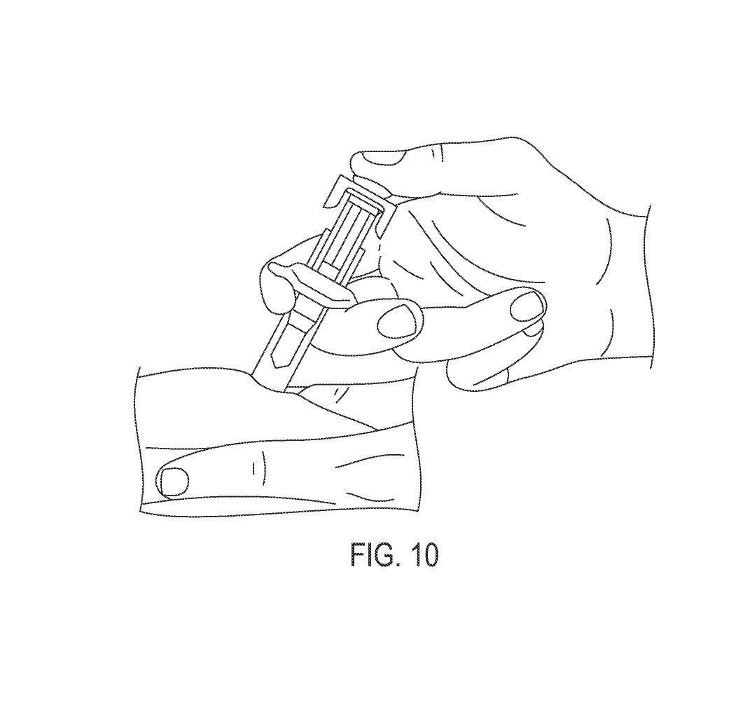

In use, a user can administer a drug or medication contained in the injection device of the present application using only one hand. For instance, referring to FIG. 10, a user may grip the flange extender 200 by placing a finger on the distal side 210, 212 of each flange extender piece 202, 204, and a thumb on the plunger 104. The user may then push the plunger 104 toward the flange extender 200, which forces the liquid drug to be expelled through the needle (not shown).

In some instances, a syringe may require aspiration before administering the medication. When aspirating the syringe, a user grips the flange extender 200 by placing two fingers of one hand on the proximal sides 206, 208 of each flange extender piece 202, 204, and a placing second hand on the plunger 104. The user may then first draw back the plunger 104 in a direction away from the flange extender 200 to create a negative pressure in the syringe and thus draw a liquid drug into the syringe. The user can then administer the liquid drug using only one hand by gripping the flange extender 200 by placing one finger on the distal side 210, 212 of each flange extender piece 202, 204, and a thumb on the plunger 104. The user may then push the plunger 104 toward the flange extender 200, which forces the liquid drug to be expelled through the needle.

The flange extender 200 is assembled onto the injection device 100 by attaching the first and second flange extender pieces 202, 204 directly to each other around the needle guard 102 and engaging the locking mechanism to lock the flange extender 200 into place. The flange extender 200 is not slid over an end of the injection device, which may contaminate the needle by hitting the needle shield or cause the needle to bend or break. The flange extender 200 can only be assembled and locked in one manner, thereby preventing incorrect orientation.

The flange extender 200 can be manufactured by using a mold cavity to form the first and second flange extender pieces 202, 204. This embodiment takes advantage of the fact that in some embodiments, such as shown in FIGS. 2-4, the first and second flange extender pieces are identical to each other. The use of a single mold for both flange extender pieces reduces manufacturing costs. The mold includes a first feature for forming a first locking mechanism, such as a locking finger, and a second feature forming a second locking mechanism to mate with the first locking mechanism, such as an aperture for receiving the locking finger, for example as shown in FIGS. 2-4. The molding process may take the form a two shot molding process. This two shot molding process includes a first step of molding the flange extender piece 202 and 204 (e.g., injecting a polycarbonate material into the mold), then rotating the mold, and then molding the soft material 211 of the flange extender piece onto the polycarbonate material. The two materials are selected such they chemically bond to each other. An advantage of this process is that no additional manufacturing steps are required to create the flange extender. In other embodiments, the flange extender 200 may be manufactured using more than one mold. In that case, a chemical or mechanical bond may hold the two flange extender pieces 202, 204 together.

While a number of exemplary aspects and embodiments have been discussed above, those of skill in the art will recognize that still further modifications, permutations, additions and sub-combinations thereof of the features of the disclosed embodiments are still possible. It is therefore intended that the following appended claims and claims hereafter introduced are interpreted to include all such modifications, permutations, additions and sub-combinations as are within their true spirit and scope.

1. A flange extender for attachment to an injection device, the flange extender comprising:
first and second flange extender pieces;
a locking mechanism to lock the first and second flange extender pieces to one another;
the first and second flange extender pieces extending in a direction transverse to a main axis of the injection device to thereby provide a surface area for manual manipulation of the injection device during an injection; and
the first and second flange extender pieces attaching to opposite sides of the injection device.

2. The flange extender of claim 1 wherein the locking mechanism comprises each of the first and second flange extender pieces having a locking finger extending from the flange extender piece and each of the first and second flange extender pieces having an aperture located within the flange extender piece adapted to receive a locking finger.

3. The flange extender of claim 1 wherein the locking mechanism comprises two locking fingers extending from the first flange extender piece and two apertures adapted to receive the locking fingers located within the second flange extender piece.

4. The flange extender of claim 1 wherein the locking mechanism comprises adhesive.

5. The flange extender of claim 1 wherein the locking mechanism comprises welding of the first flange extender piece to the second flange extender piece.

6. The flange extender of claim 1 wherein the first flange extender piece is connected to the second flange extender piece by a loop and hook mechanism.

7. The flange extender of claim 1 wherein the first and second flange extender pieces have the same physical configuration.

8. The flange extender of claim 1 wherein the first and second flange extender pieces define a longitudinal axis substantially transverse to the main axis of the injection device, and wherein the fingers extend in a direction parallel to the longitudinal axis of the flange extender when the first flange extender piece is locked to the second flange extender piece.

9. The flange extender of claim 1 wherein the first and second flange extender pieces each include a proximal side and a distal side, and wherein the first piece and the second piece each have a soft material on the distal side.

10. The flange extender of claim 9 wherein first and second flange extender pieces each include a curved surface on the distal side.

11. The flange extender of claim 1 wherein the first and second flange extender pieces each include a rounded peripheral end.

12. The flange extender of claim 1 wherein when the two flange extender pieces are connected a recess is formed, the recess having a rectangular shape.

13. The flange extender of claim 1 wherein when the two flange extender pieces are connected a recess is formed, the recess having a circular shape.

14. The flange extender of claim 1 wherein when the two flange extender pieces are connected a recess is formed, the recess having a cut flange shape.

15. The flange extender of claim 1 further comprising a feature for preventing movement of the flange extender in the distal direction.

16. A flange extender for attachment to an injection device, the flange extender comprising:
first and second flange extender pieces adapted to attach to opposite sides of the injection device, each piece having a locking finger extending from the piece and an aperture adapted to receive the locking finger and lock the first and second flange extender pieces to one another;
wherein the first and second flange extender pieces are of identical construction whereby each locking finger and aperture are spaced and configured such that the locking finger of the first flange extender piece locks into the aperture of the second flange extender piece and the locking finger of the second flange extender piece locks to the aperture of the first flange extender piece to thereby securely attach the first and second flange extender pieces to the injection device.

17. The flange extender of claim 16 wherein the first and second flange extender pieces include a recess between the locking finger and the aperture to accommodate the injection device.

18. The flange extender of claim 16 wherein the flange extender is formed as a single molded part comprising two materials co-molded with each other.

19. An injection assembly comprising:
a pre-filled syringe having a barrel and a plunger;
a needle guard surrounding the barrel of the syringe, the needle guard being movable between a retracted position and an extended position;
a housing for the needle guard having a finger flange; and
a flange extender comprising a first flange extender piece and a second flange extender piece located around the housing;
wherein the first flange extender piece is connected to the second flange extender piece to thereby lock the flange extender around the housing.

20. The injection assembly of claim 19 wherein the flange extender surrounds the finger flange of the housing.

21. The injection assembly of claim 19 wherein the flange extender is placed around the housing at a position rotated 90 degrees from the finger flange.

22. The injection assembly of claim 19 wherein the first flange extender piece is snap-fit to the second flange extender piece.

23. The injection assembly of claim 19 wherein the first flange extender piece is connected to the second flange extender piece by adhesive.

24. The injection assembly of claim 19 wherein the first flange extender piece is welded to the second flange extender piece.

25. The injection assembly of claim 19 wherein the first flange extender piece is connected to the second flange extender piece by a loop and hook mechanism.

26. The injection assembly of claim 19 wherein each of the first and second flange extender pieces have a locking finger extending from the piece and each of the first and second flange extender pieces have an aperture adapted to receive a locking finger and lock the first and second flange extender pieces to one another;
the first and second flange extender pieces extending in a direction transverse to a main axis of the injection device to thereby provide a surface area for manipulation of the injection device during an injection; and
the first and second flange extender pieces attaching to opposite sides of the flange of the injection device.

27. The injection assembly of claim 19 wherein each of the first and second flange extender pieces are adapted to attach to opposite sides of the finger flange of the housing, each piece having a locking finger extending from the piece and an aperture adapted to receive the locking finger and lock the first and second flange extender pieces to one another;
wherein the first and second flange extender pieces are of identical construction whereby the locking finger and aperture are spaced and configured such the locking finger of the first flange extender piece locks into the aperture of the second flange extender piece and the locking finger of the second flange extender piece locks to the aperture of the first flange extender piece to thereby securely attach the flange extender pieces to the housing of the injection assembly.

28. A method of manufacturing a flange extender for an injection device comprising:
using a mold cavity to form first and second flange extender pieces, the first and second flange extender pieces adapted to lock onto an injection device, wherein the mold includes a first feature forming a first locking mechanism and a second feature forming a second locking mechanism for mating with the first locking mechanism.

29. The method of claim 28 wherein the first locking mechanism is a locking finger and the second locking mechanism is an aperture.

30. The method of claim 28 wherein the first and second flange extender pieces are essentially identical in construction, and wherein the locking finger of the first flange extender piece locks into a corresponding aperture of the second flange extender on the second flange extender piece and the locking finger of the second flange extender piece locks to the aperture of the first flange extender piece to thereby securely attach the first and second flange extender pieces to the injection device.

31. A method of assembling an injection device comprising:
providing a syringe;
directly attaching opposing pieces of a flange extender over the syringe; and
engaging a locking mechanism to lock the flange extender around the syringe.

32. The method of claim 31 wherein the syringe has a needle guard and the flange extender locks around the needle guard.
